(19) United States
(12) Patent Application Publication
(10) Pub. No.: US 2006/0078874 A1
(43) Pub. Date: Apr. 13, 2006

(54) Use of nuclear hormone receptors

(75) Inventor: Christian Groendahl, Vaerloese (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/129,468

(22) Filed: May 13, 2005

(30) Foreign Application Priority Data
Nov. 14, 2002 (DK) PA 2002 01754
Mar. 14, 2003 (DK) PA 2003 00385

(51) Int. Cl. C12Q 1/00; A61K 31/56

(52) U.S. Cl. 435/4; 514/177

(57) ABSTRACT

The present invention presents the means to identify agonists and antagonists of the NR/ligand interaction by using NRs. The term “NR” refers to any proteins either derived from a naturally occurring NR (from any species), or which shares significant structural and functional characteristics peculiar to a naturally occurring NR. Such an NR may result when regions of a naturally occurring receptor are deleted or replaced in such a manner as to yield a protein having a similar function. Homologous sequences, allelic variations, and natural mutants; induced point, deletion, and insertion mutants; alternatively expressed variants; proteins encoded by DNA which hybridise under high or low stringency conditions to nucleic acids which encode naturally occurring NR; proteins retrieved from naturally occurring materials; and closely related proteins retrieved by antisera directed against NR. Similarly, this applies to signalling proteins.

### CROSS-REFERENCE TO RELATED APPLICATIONS

This application is a continuation of international application no. PCT/DK2003/000776 filed Nov. 12, 2003 and claims priority under 35 U.S.C. 119 of Danish applications PA 2002 01754 filed Nov. 14, 2002 and PA 2003 00385 filed Mar. 14, 2003, the contents of which are fully incorporated herein by reference.

### FIELD OF THIS INVENTION

The present invention relates to the use of NRs to identify fertility promoting compounds and contraceptives, the use of polynucleotides coding for NRs to identify fertility promoting compounds and contraceptives, the use of probes hybridising with nucleic acids encoding NRs to identify fertility promoting compounds and contraceptives, the use of DNA constructs comprising a sequence encoding NRs to identify fertility promoting compounds and contraceptives, the use of culture cell lines wherein the DNA sequence encodes NRs to identify fertility promoting compounds and contraceptives, the use of antibodies specifically binding to NRs to identify fertility promoting compounds and contraceptives, the use of hybridoma producing monoclonal antibodies specifically binding to NRs to identify fertility promoting compounds and contraceptives, and methods for detecting the presence of a compound having affinity to NRs.

### BACKGROUND OF THIS INVENTION

Since the first IVF pregnancy was delivered in 1978, this procedure has resulted in thousands of pregnancies and opened a vast new frontier of research and treatment for the infertile couples. Still, there is a significant need for improved infertility treatment modalities today. It is presumed that about one out of seven couples experience problems with sub fertility or infertility.

IVF of human oocytes has become commonly used for the treatment of female and male sub fertility. The standard IVF treatment includes a long phase of hormone stimulation of the female patient. The aspirated oocyte is subsequently fertilised in vitro and cultured. Continuous efforts have been made to optimise and simplify this procedure. Nevertheless, the overall pregnancy rate cannot be increased significantly over about 20% with the current treatment modalities. In a large European survey of IVF patients, it was found that 7.2 oocytes out of 11.5 aspirated oocytes per patient had undergone resumption of meiosis immediately before fertilisation, only 4.3 oocytes were fertilised and only 2.2 oocytes reached the 8-cell embryo stage after fertilisation and in vitro culture (ESHRE, Edinburgh, 1997).

Due to the very unpredictable quality of the state of the art embryos today, more than one embryo has to be transferred just to give a reasonable chance of success. Therefore, it is common to transfer 2-3 embryos (up to 5 embryos in some countries), which carries the very large side effect of multiple pregnancies with great discomfort and risk to both patient and children. Moreover, it has been estimated that the increased health care expenses due to multiple birth (twins, triplets etc.) is exceeding the entire IVF expenses.

Hence, there are several disadvantages with the current treatment.

Furthermore, weight gain, bloating, nausea, vomiting, labile mood and other patient discomforts together with patient reluctance to inject themselves are reported as disadvantages.

Consequently, at present, in vitro maturation in humans has proven highly unsuccessful despite substantial interest and clinical efforts.

It is known from WO 96/00235 that certain sterol derivatives can be used for regulating meiosis. An example of such a sterol is 4,4-dimethyl-5α-cholesta-8,14,24-triene-3β-ol (hereinafter designated FF-MAS).

Nuclear Hormone Receptors (herein designated NRs) are encoded by a superfamily of homologous genes and information about them can, for example, be found at the web page: http://www.ens-lyon.fr/LBMC/laudet/nurebase.html. The NR protein superfamily is caracterized by conserved domains: the DNA-binding domain and the ligand-binding domain. Splice variants, for example, without the DNA binding domain could act through a cytoplasmic cofactor and induce rapid signals such as increased levels of second messengers including calcium and cAMP as well as activation of phospholipase C. An example is the estrogen receptor and the fast and transient activation of MAPK signaling cascade (Bjornstrom L & Sjoberg M: Signal transducers and activators of transcription as downstream targets of nongenomic estrogen receptor actions. Mol. Endocrinol. 2002 16 p. 2202-2214). The NRs are well studied and involved in various physiological functions, as well as numerous pathologies, including diabetes, obesity, cancer, or metabolic disorders (Giguere V.: Structure and function of the nuclear receptor superfamily for steroid, thyroid hormone and retinoic acid. Genet. Eng. (N Y) 12 (1990), 183-200, and Mangelsdorf D J et al.: The nuclear receptor superfamily: the second decade. Cell. 1995 83(6): 835-839). The activity of NRs can be controlled by at least four distinct mechanisms: 1) binding of a small lipophilic ligand by the receptor or its partner in heterodimer complexes; 2) covalent modification, usually in the form of phosphorylation regulated by events at the cellular membrane or during the cell cycle; 3) protein-protein interactions, generally through contacts with other transcription factors including nuclear receptors themselves; and finally 4) some nuclear receptors mediate nongenomic effects that are too rapid to involve changes in gene transcription. All mechanisms can either work individually or in concert with each other to modulate a specific signal. Some of the NRs that are able to mediate the nongenomic effects are the estrogen receptor and the progesteron receptor which both are involved in regulating not only events in the reproductive tissues but also in for instance the cardiovascular system.

The Liver-X-Receptors (herein designated LXRs) are members of the nuclear receptor (NR) superfamily. The activity of these receptors can be controlled by at least four distinct mechanisms: 1) binding of a small lipophilic ligand by the receptor or its partner in heterodimer complexes; 2) covalent modification, usually in the form of phosphorylation regulated by events at the cellular membrane or during the cell cycle; 3) protein-protein interactions, generally through contacts with other transcription factors including nuclear receptors themselves; and finally 4) some nuclear receptors mediate non-genomic effects that are too rapid to involve changes in gene transcription. All mechanisms can either work individually or in concert with each other to modulate a specific signal. Some of the NRs that are able to mediate the nongenomic effects are the estrogen receptor and the progesteron receptor which both are involved in regulating not only events in the reproductive tissues but also in for instance the cardiovascular system. The LXRs bind and are activated by oxysterols such as 22R-hydroxycholesterol and are known to regulate the sterol catabolism and thus, are key sensors for maintaining cholesterol homeostasis (reviewed in Peet et al: The LXRs: a new class of oxysterol receptors. Curr. Opin. Genet. Dev. 8 (1998), 571-575). The theory so far has been that binding of oxysterols to LXR proteins causes recruitment of co-factors which again stimulates transcription of genes encoding proteins involved in cholesterol metabolism. Non-genomic actions of the NR ligands include the rapid actions of for instance estradiol that increases cAMP concentrations (Szego & Davis: Adenosine 3′,5′-monophosphate in rat uterus: acute elevation by estrogen. Proc Natl Acad Sci U S A. 1967 58:1711-8). Thus, the effects of oxysterols are manifold and it is possible that one effect measured at a certain time in a certain cell line differ from effects in other cell types.

Meiotic regulating substances (hereinafter designated MASs (and MAS in the singular)) constitute active signalling molecules first identified in follicular fluid and in bull testicular tissue. Some MASs are described in Nature 374 (1995), 559-562, and in Biol. Reprod. 58 (1998), 1297 et seq., and MASs also comprise all the compounds of the general formula I, Ia, Ib, and Ic mentioned in any of the international patent applications having the international publication number WO 96/00235 (our ref.: 4228), WO 96/27658, WO 97/00884 (our ref.: 4475), WO 98/28323 (our ref.: 5141), WO 99/32506 (earliest priority: 1997.12.18), WO 98/52965 (earliest priority: 1997.05.16), WO 98/54965, WO 98/55498, WO 99/67273 (our ref.: 5558), WO 99/58549 (our ref.: 5509), WO 2000/47604 (our ref.: 5769), WO 2000/68245 (our ref.: 6238), and WO 2001/62771 (our ref.: 6239), preferably all the specific compounds mentioned specifically in said WO specifications covered by said formula, more specifically in claim 1 thereof. MASs are potent activators of the meiotic process.

In addition to its meiosis regulating effect, FF-MAS is also a part of the cholesterol biosynthesis pathway, which prompted Janowski et al. to investigate the ability of FF-MAS to activate LXRα (Janowski et al. Nature 383 (1996), 728-731). FF-MAS was able to transactivate LXRα but no direct binding was explored. Thus, the FF-MAS-induced activation of LXRα could be caused by an indirect effect. However, no correlation between LXR activators in vitro and meiosis activation in vivo has ever been seen. (Grondahl et al., Meiosis-activating sterol promotes resumption of meiosis in mouse oocytes cultured in vitro in contrast to related oxysterols, vide Biol. Reprod. 58 (1998), 1297-1302).

The Liver-X-receptor (LXR) superfamily contains two members, LXRα and LXRβ. LXRα is activated by oxidized forms of cholesterol such as 22(R)-hydroxycholesterol (vide Nature 383 (1996), 728-731). No significant differences in the ability of LXRα or LXRβ to respond to oxysterols have been reported. Human LXRα has the potential to function as a ligand-dependent transcription factor when complexed with its hetero-dimeric partner, the retinoid-X receptor (RXR), vide Genes Dev. 9 (1995), 1033-1045. LXRα is most highly expressed in the liver, brain, placenta, small intestine, adipose tissue, and macrophages, whereas LXRβ is ubiquitously expressed. Both forms are expressed in mouse oocytes. The principle physiological role of LXRα and LXRβ appears to be the removal of excess cholesterol via transcription of genes encoding proteins involved in the cholesterol biosynthesis pathway. LXRβ is described in Proc. Natl Acad. Sci. USA 91 (1994), 10809-10813.

One way of trying to find compounds which effectively regulate meiosis is the use of pertinent receptors.

Receptors are defined as proteinaceous macromolecules that perform a signal transducing function upon ligand binding. Many receptors are located on the outer cell membrane, others are located intracellularly. The substance which is bound by the receptor is called a ligand, a term which is definitionally meaningful only in terms of its counterpart receptor. The term “ligand” does not imply any particular molecular size or other structural or compositional feature other than that the substance in question is capable of binding, cleaving, or otherwise interacting with the receptor in such a way that the receptor conveys information about the presence of the ligand to a target molecule. Stated alternatively, not all substances capable of binding a receptor are ligands, but all ligands are capable of binding a receptor. Receptors do not include such substances as immunoglobulins.

Receptors are believed to function by a process variously termed activation or signal transduction. A ligand binds to the ligand binding domain in such a way that the conformation of the receptor molecule changes. This conformational change, called activation, modifies the effect of the receptor on cytoplasmic components. Among changes brought about by receptor activation are changes in or development of receptor enzymatic activity.

Signalling proteins such as cAMP, IP3, kinases, and phosphatases are proteins ubiquitously found in all tissues. These proteins cascade by various pathways, the stimulus from ligand/receptor interaction down stream to cellular events, typically changing the enzymatic activity or functional state of effector molecules.

It is an object herein to provide readily reproducible, simple assay systems that can be practiced on a large scale for determining not only ligand binding but also the character of the binding as agonistic or antagonistic.

Similarly, meaningful clinical diagnosis often depends upon the assay of biologically active ligand without interference from inactive forms of the ligand, for example, ligands that have been subject to enzymatic or other processes in the test subject that change or even eliminate the activity of the ligand. Immunoassay methods are widely used in determining ligands in test samples. However, it is often quite difficult to identify antibodies that are able to discriminate between the active and inactive forms of a ligand. Receptors have frequently been used in place of antibodies as analyte binding reagents. However, not all substances that bind to receptors are necessarily capable of inducing receptor activity, i.e., active biologically.

It is an object herein to provide a method that will identify ligands in clinical test samples which are active in inducing or inhibiting signal transduction by their receptors.

Cytoplasmic proteins can act as receptors or signalling molecules in cascading the stimulus from the ligand to cellular events. Various receptors or signalling protein types make use of different path ways (for example small G proteins, calcium fluxes, phospatases, and lipases), all of them resulting in changes of enzymatic activity or gene transcription.

It would also be desirable to screen and develop new agonists and/or antagonists specific for an NR for the use of antiinfertility or contraception drugs, but to date this has not been possible. Quite surprisingly, the present invention fulfils these and other related needs.

One Object of This Invention is to Improve the Prior Art.

Another object of this invention is to elucidate the reaction mechanism involved in meiosis of oocytes.

A still further object of this invention is to furnish a method by which compounds which can conveniently be used to regulate meiosis can be found.

A further object of this invention is to furnish a method by which compounds which can conveniently be used as pro-fertility treatment can be found.

A further object of this invention is to furnish a method by which compounds which can conveniently be used as contraceptives can be found.

A further object of this invention is to furnish a method by which compounds which can conveniently be used to treat menopause patients can be found.

A further object of this invention is to find the receptor which is a signalling partner for FF-MAS.

A further object of this invention is to find a receptor which is involved in maturation of oocytes.

A further object of this invention is to find receptors which are important in controlling pathways in the reproduction.

### DEFINITIONS

Briefly, herein an NR (NRs in plural) comprises NRs from any species and similar peptides having an amino acid homology (or identity) of at least 80%, preferably at least 90%, more preferred at least 95%. NRs are involved in the male and female meiosis. More specifically, NRs are involved in the regulation of the oocyte maturation. Even more specifically, NRs are the FF-MAS receptor or a FF-MAS signalling protein. Examples of specific NRs are LXRα and LXRβ.

The HTRF assay is described in Methods 25 (2001), 54-61. (HTRF is an abbreviation for homogeneous time-resolved fluorescence.)

### SUMMARY OF THIS INVENTION

It has now, surprisingly, been found that NRs can be used to identify compounds having ability to regulate meiosis. Furthermore, NRs can be used to identify compounds having cholesterol lowering effect.

Now, NRs have been shown to be involved in the gamete maturation process induced by FF-MAS, specifically inducing, upon ligand activation, germinal vesicle breakdown (hereinafter designated GVBD) in mouse oocytes cultured in vitro.

An NR is any protein related to any of the NRs, for example, LXRα or LXRβ, that possesses the same functional characteristic regarding the interaction with FF-MAS or other endogenous meiosis activating sterols, for example, 3α-hydroxycholest-8,14-diene; 3α-hydroxy-4,4-dimethylcholest-8,24-diene; and 3α-hydroxycholest-8,24-diene, or their metabolites (as ligand). Functional characteristics include binding, receptor activation, and subsequent GVBD in oocytes.

The NR can be used to discover profertility and antifertility compounds which can be used to treat men and women.

Cells or bacteria which express the NRs may also be used to identify compounds which can alter the NR-mediated metabolism of a cell. Compounds may be screened for binding to the NR, and/or for effecting a change in receptor or signalling protein-mediated metabolism in the host cell. Agonists and/or antagonists of the NRs may also be screened in cell-free systems using purified NRs or binding fragments thereof for the effect on ligand/receptor interaction or ligand/signalling protein interaction, or using reconstituted systems such as micelles which also provide the ability to assess metabolic changes.

In a related embodiments, the invention relates to methods for diagnosis, where the presence of mammalian NRs in a biological sample may be determined. For example, a monospecific antibody which specifically binds the receptor or signalling protein is incubated with the sample under conditions conducive to immune complex formation, which complexes are then detected, typically by means of a label such as an enzyme, fluorophore, radionuclide, chemiluminiscer, particle, or a second labelled antibody. Thus, means are provided for immunohistochemical staining of tissues, including ovarian or testicular tissues, for the subject NR.

### BRIEF DESCRIPTION OF THE FIGURES

FIGS. 1A and 1B are graphs showing that FFMAS behaves like an agonist for human LXRs.

FIG. 2 is a graph showing that FFMAS behaves like a partial agonist for human FXRalpha.

FIG. 3 is a graph showing that FFMAS behaves like a partial agonist for mouse FXRbeta.

FIG. 4 shows visualization by immunohistochemistry of LXRα in the mouse oocyte.

### DETAILED DESCRIPTION OF THIS INVENTION

The present invention presents the means to identify agonists and antagonists of the NR/ligand interaction by using NRs. The term “NR” refers to any proteins either derived from a naturally occurring NR (from any species), or which shares significant structural and functional characteristics peculiar to a naturally occurring NR. Such an NR may result when regions of a naturally occurring receptor are deleted or replaced in such a manner as to yield a protein having a similar function. Homologous sequences, allelic variations, and natural mutants; induced point, deletion, and insertion mutants; alternatively expressed variants; proteins encoded by DNA which hybridise under high or low stringency conditions to nucleic acids which encode naturally occurring NR; proteins retrieved from naturally occurring materials; and closely related proteins retrieved by antisera directed against NR. Similarly, this applies to signalling proteins.

By NR “ligand” is meant a molecule capable of being bound by the ligand-binding domain of NR, an NR analogue, or chimeric NR as generally described in U.S. patent specification No. 4,859,609, hereby incorporated by reference herein. The molecule may be chemically synthesised or may occur in nature. Ligands may be grouped into agonists and antagonists. Agonists are those molecules whose binding to a receptor induces the response pathway within a cell. Antagonists are those molecules whose binding to a receptor blocks the response pathway within a cell.

By “isolated” NRs is meant to refer to NRs which is in other than its native environment such as a mammalian oocyte, including, for example, substantially pure NR as defined herein below. More generally, isolated is meant to include NRs as a heterologous component of a cell or other system. For example, NRs may be expressed by a cell transfected with a DNA construct which encodes NRs, separated from the cell and added to micelles which contain other selected receptor or signalling proteins. In some instances, the term NR covers both an NR and an NR signalling protein.

In another aspect, the invention provides means for investigating the NR/ligand interaction, and thus treating, therapeutically and/or prophylactically, a disorder which can be linked directly or indirectly to NRs or to its ligands, such as FF-MAS. By virtue of having NR, agonists or antagonists may be identified which stimulate or inhibit, respectively, the interaction of ligand with NR. With either agonists or antagonists, the metabolism and reactivity of cells which express NR are controlled, thereby providing a means to control meiosis in order to treat infertility or to achieve a novel principle of contraception.

Thus, the invention provides screening procedures for identifying agonists or antagonists of events mediated by the ligand/NR interaction. Such screening assays may employ a wide variety of formats, depending to some extent on which aspect of the ligand, receptor or signalling protein interaction is targeted. For example, such assays may be designed to identify compounds which bind to the NR and thereby block or inhibit interaction of the NR with the ligand. Other assays can be designed to identify compounds which can substitute for ligand and therefore stimulate NR-mediated intracellular pathways. Yet other assays can be used to identify compounds which inhibit or facilitate the association of NRs to FF-MAS and thereby mediate the cellular response to NRs ligand.

In one functional screening assay, the initiation of fertilisation activation events are monitored in eggs which have been injected with, for example, mRNA which codes for NRs and subsequently exposed to selected compounds which are being screened, in conjunction with or apart form an appropriate ligand. See generally, Kline et al., Science 241 (1988), 464-467, incorporated herein by reference.

The screening procedure can be used to identify reagents such as antibodies which specifically bind to NR and substantially affect its interaction with ligand, for example. The antibodies may be monoclonal or polyclonal, in the form of antiserum or monospecific antibodies, such as purified antiserum or monoclonal antibodies or mixtures thereof. For administration to humans, for example, as a component of a composition for in vivo diagnosis or imaging, the antibodies are preferably substantially human to minimise immunogenicity and are in substantially pure form. By substantially human is meant NR generally containing at least about 70% human antibody sequence, preferably at least about 80% human, and most preferably at least about 90-95% or more of a human antibody sequence to minimise immunogenicity in humans.

In another aspect the invention concerns diagnostic methods and compositions. By means of having the NRs molecule and antibodies thereto, a variety of diagnostic assays are provided. For example, with antibodies, including monoclonal antibodies, to NRs, the presence and/or concentration of NR in selected cells or tissues in an individual or culture of interest may be determined. These assays can be used in the diagnosis and/or treatment of diseases such as, for example, male infertility, female infertility, or by means of contraception in both gender.

Kits can also be supplied for use with the NR in the detection of the presence of the NR or antibodies thereto, as might be desired in the case of autoimmune disease. Thus, antibodies to NRs, preferably monospecific antibodies such as monoclonal antibodies, or compositions of the NR may be provided, usually in lyophilised form in a container, either segregated or in conjunction with additional reagents, such as anti-antibodies, labels, gene probes, polymerase chain reaction primers and polymerase, and the like.

Furthermore, the present invention relates to the use of an isolated antibody which specifically binds to an NR. In this isolated antibody said antibody may be a monoclonal antibody. This isolated antibody may block the binding of MAS to an NR.

Furthermore, the present invention relates to the use of a hybridoma which produces a monoclonal antibody as mentioned herein.

Furthermore, the present invention relates to a method for detecting the presence of a compound which has affinity for an NR, comprising the steps of a) contacting the compound with the NR, a peptide fragment thereof or a salt thereof; and b) measuring the affinity of said compound for the NR. This method for detecting the presence of MAS antagonists, may comprise the steps of a) exposing a compound in the presence of a MAS agonist including MAS (FF-MAS) to an NR coupled to a response pathway under conditions and for a time sufficient to allow binding of the compound to the NR and an associated response through the pathway; and b) detecting a reduction in the stimulation of the response pathway resulting from the binding of the compound to the NR, relative to the stimulation of the response pathway by the MAS agonist alone and there from determining the presence of a MAS antagonist. Furthermore, a method for detecting the presence of MAS agonists, may comprise the steps of a) exposing a compound in the presence of a MAS antagonist to an NR coupled to a response pathway under conditions and for a time sufficient to allow binding of the compound to the NR and an associated response through the pathway; and b) detecting an increase of the stimulation of the response pathway resulting from the binding of the compound to the NR, relative to the stimulation of the response pathway by the MAS antagonist alone and there from determining the presence of a MAS agonist.

Furthermore, the present invention relates to a compound or a salt thereof which has affinity for the NR and which compound or salt is detected by a method described herein.

Furthermore, the present invention relates to a kit for screening a compound or a salt thereof which has affinity for an NR, which contains the NR, the peptide fragment thereof or a salt thereof.

Furthermore, the present invention relates to a method of screening for ligands to the NR, i.e., agonists or antagonists of FF-MAS activity, the method comprising incubating an NR as defined herein with a substance suspected to be an agonist or antagonist of FF-MAS, and subsequently with FF-MAS, or an analogue thereof, and detecting any effect of binding of FF-MAS, or the analogue to the NR. Alternatively, the method of screening for ligands to the NR, i.e., agonists or antagonists of FF-MAS activity, may comprise incubating FF-MAS, or an analogue thereof with a substance suspected to be an agonist or antagonist of activity of FF-MAS, and subsequently with an NR as described herein, and detecting any effect of binding of FF-MAS, or the analogue to the receptor.

Furthermore, the present invention relates to the use of an NR as defined herein for screening for agonists or antagonists of activity of FF-MAS.

Furthermore, the present invention relates to a method for identifying a compound having meiosis activating activity, said method comprising the following steps: a) testing the effect of said compound in an HTRF assay as defined in the above specification and selecting the compound if is has an EC₅₀ value above about 500 nM and b) testing the effect of the selected compound in an oocyte maturation assay, for example as described in Example 1 below, and selecting said compound if the GVBD value is above about 70, preferably above about 85.

Furthermore, the present invention relates to a method for identifying a compound having meiosis activating activity, said method comprising the following steps: a) testing the effect of said compound in an HTRF assay as defined in the above specification and selecting the compound if is has an EC₅₀ value above about 500 nM and b) testing the effect of the selected compound in an oocyte maturation assay, for example as described in Example 2 below, and selecting said compound if the GVBD value is below about 50, preferably below about 35.

Furthermore, the present invention relates to the use of DNA constructs as defined herein for isolation of tissue and/or organ specific variants of the NR.

Furthermore, the present invention relates to the use of an NR isolated as described herein.

The mentioning herein of a reference is no admission that it constitutes prior art. Herein the word “comprise” is to be interpreted broadly meaning “include”, “contain” or “comprehend” (vide, for example EPO guidelines C 4.13). All articles referred to herein are hereby incorporated by reference.

The features described in the foregoing description and in the following may, both separately and in any combination thereof, be material for realizing this invention in diverse forms thereof.

The present invention is further described in the following examples which, however, are not to be construed as limiting.

### EXAMPLE 1

An Agonistic Oocyte Assay Can be Performed as Follows:

Oocytes were obtained from immature female mice (C57BL/6J×DBA/2J F1-hybrid, Bomholtgaard, Denmark) weighing 13-16 grams, that were kept under controlled temperature (20-22° C.), light (lights on 06.00-18.00) and relative humidity (50-70%). The mice received an intra-peritoneal injection of 0.2 ml gonadotropins (Gonal-F, Serono) containing 20 IU FSH and 48 hours later the animals were killed by cervical dislocation.

The ovaries were dissected out and the oocytes were isolated in Hx-medium (see below) under a stereo microscope by manual rupture of the follicles using a pair of 27 gauge needles. Spherical oocytes displaying an intact germinal vesicle (hereinafter designated GV) were divided in cumulus enclosed oocytes (hereinafter designated CEO) and naked oocytes (hereinafter designated NO) and placed in α-minimum essential medium (α-MEM without ribonucleosides, Gibco BRL, Cat. No. 22561) supplemented with 3 mg/ml bovine serum albumin (BSA, Sigma Cat. No. A-7030), 5 mg/ml human serum albumin (HSA, Statens Seruminstitute, Denmark), 0.23 mM pyruvate (Sigma, Cat. No S-8636), 2 mM glutamine (Flow Cat. No. 16-801), 100 IU/ml penicillin and 100 μg/ml streptomycin (Flow, Cat No. 16-700). This medium was supplemented with 3 mM hypoxanthine (Sigma Cat. No. H-9377) and designated Hx-medium.

The oocytes were rinsed three times in Hx-medium and oocytes of uniform size were divided into groups of CEO and NO. CEO and NO were cultured in 4-well multidishes (Nunclon, Denmark) in which each well contained 0.4 ml of Hx-medium. One control well (i.e., 35-45 oocytes cultured in identical medium with no addition of test compound) was always cultured simultaneously with 3 test wells (35-45 oocytes per well supplemented with test compound).

The oocytes were cultured in a humidified atmosphere of 5% CO₂ in air for 24 hours at 37° C. By the end of the culture period, the number of oocytes with germinal vesicle (hereinafter designated GV), GVBD, and polar body (herein designated PB), respectively, were counted using a stereomicroscope (Wildt, Leica MZ 12). The percentage GVBD, defined as percentage of oocytes undergoing GVBD per total number of oocytes in that well, was calculated as:
% GVBD=100×(number of GVBD+number of PB)/(total number oocytes).

The % PB was defined as percentage of oocytes displaying one extruded polar body per total number of oocytes in that well.

The effect of the tested compounds has been indexed against control level and FF-MAS where controls and FF-MAS are indexed to an effect of 0 and 100, respectively. The relative effect of the tested compound is calculated as follows:
Relative effect=100×((test GVBD %−negative control GVBD %)/(FF-MAS GVBD %−negative control GVBD %)).

Results

TABLE 1
The mean percentage GVBD, the mean percentage PB and
mean Relative Effect of a control and FF-MAS after culture
of naked oocytes (NO) in vitro for 24 hours.

| | Concentration; | Mean; | Mean; | Mean |
|---|---|---|---|---|
| | μM | % GVBD | % PB | Relative Effect |
| Control | 0 | 11.9 | 5.5 | 0 |
| FF-MAS | 10 | 86.1 | 28.5 | 100 |

### EXAMPLE 2

An Antagonistic Oocyte Assay Can be Performed as Follows:

Animals

Oocytes were obtained from immature female mice (C57BI/6J×DBA/2J F1-hybrids, Bomholtgaard, Denmark) weighing 13-16 grams, that were kept under controlled lighting and temperature. The mice received an intra-peritoneal injection of 0.2 ml gonadotropins (Gonal F, Serono, Solna, Sweden, containing 20 IU FSH, alternatively, Puregon, Organon, Swords, Ireland containing 20 IU FSH) and 48 hours later the animals were killed by cervical dislocation.

Test of Meiosis-Inhibiting Substances in the Oocyte Test

The ovaries were dissected out and the oocytes were isolated in Hx-medium (see below) under a stereo microscope by manual rupture of the follicles using a pair of 27 gauge needles. Spherical, naked oocytes (NO) displaying an intact germinal vesicle (GV) were placed in α-minimum essential medium (α-MEM without ribonucleosides, Gibco BRL, Cat. No. 22561) supplemented with 3 mM hypoxanthine (Sigma Cat. No. H-9377), 8 mg/ml human serum albumin (HSA, Statens Seruminstitut, Denmark), 0.23 mM pyrubate (Sigma, Cat. No. S-8636), 2 mM glutamine (Flow Cat. No. 16-801), 100 IU/ml penicillin and 100 μg/ml streptomycin (Flow, Cat No. 16-700). This medium was designated Hx-medium.

Naked oocytes (NO) were rinsed three times in Hx-medium. FF-MAS has previously been shown to induce meiosis in NO in vitro (Byskov, A. G. et al. Nature 374 (1995), 559-562). NO were cultured in Hx-medium supplemented with 5 μM FF-MAS in co-culture with the test compounds in different concentrations in 4-well multidishes (Nunclon, Denmark) in which each well contained 0.4 ml of the medium and 35-45 oocytes. One positive control (i.e., 35-45 oocytes cultured in Hx-medium containing FF-MAS with no addition of test compound) was always run simultaneously with the test cultures, which were supplemented with different concentrations of the compounds to be tested. In addition, one negative control (35-45 oocytes cultured in Hx-medium alone) was run simultaneously with the positive control.

Examination of Oocytes

By the end of the culture period, the number of oocytes with GV or GVBD and those with PB was counted using a stereomicroscope or an inverted microscope with differential interference contrast equipment. The percentage of oocytes with GVBD+PB per total number of oocytes were calculated in the test cultures and in the control (positive and negative) culture groups. The relative inhibition of the test compound was calculated by the following formula:
Inhibition of test compound (in percentage)=100−[(GVBD_{test compound}−GVBD_{negative control})×100/(GVBD_{positive control}−GVBD_{negative control})].

In case of a dose response curve, an IC₅₀ (dose, which lead to a 50% inhibition) was calculated.

### EXAMPLE 3

An agonist designated 90-8684 showed activity against LXRα, LXRβ, and PXR.

90-8684 is described in J. Med. Chem. 45 (2002), 1963-66, and has the systematic name: 2-(3-{3-[[2-chloro-3-(trifluoromethyl)benzyl](2,2-diphenylethyl)amino]propoxy}phenyl)acetic acid and the formula:

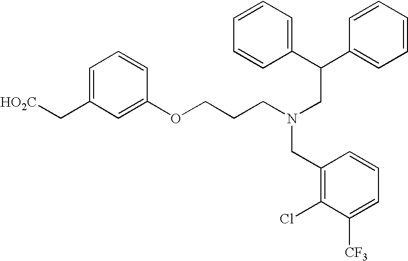

This compound is also mentioned in WO 02/24632, the latter mentioning other interesting compounds which can be used according to the present invention.

Testing 90-8684 for meiosis-inducing effects we found in an oocyte maturation assay a dose-dependent maturation of the oocytes, indicating that NR agonists indeed is able to regulate meiosis in mouse oocytes.

Results:

90-8684 in NkO Test:

| | % GVBD + PB | Σ oocytes |
|---|---|---|
| Hx | 32 ± 3% | 135 |
| Hx + 5 μM 90-8684 | 45 ± 5% | 69 |
| Hx + 10 μM 90-8684 | 49 ± 8% | 149 |
| Hx + 15 μM 90-8684 | 54 ± 5% | 144 |
| Hx + 10 μM FF-MAS | 90 ± 8% | 159 |

90-8684 in CEO Test:

| | % GVBD + PB | Σ oocytes |
|---|---|---|
| Hx | 33 ± 5% | 80 |
| Hx + 2.5 μM 90-8684 | 69 ± 3% | 82 |
| Hx + 5 μM 90-8684 | 71 ± 6% | 78 |
| Hx + 10 μM 90-8684 | 86 ± 7% | 165 |
| Hx + 10 μM FF-MAS | 78 ± 9% | 158 |

### EXAMPLE 4

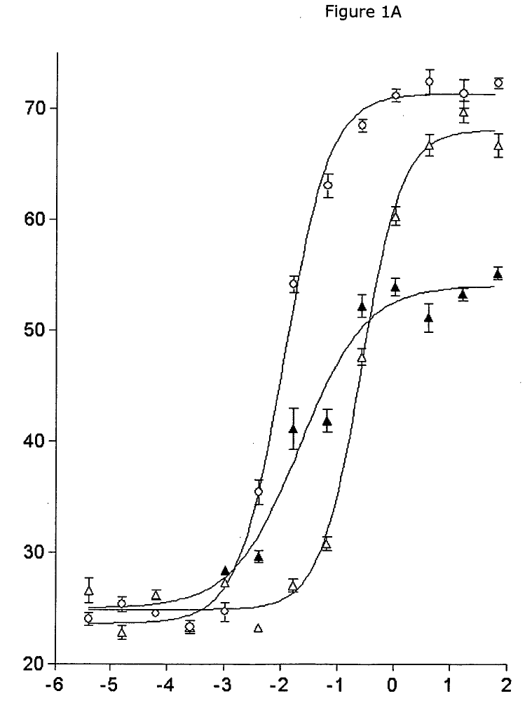

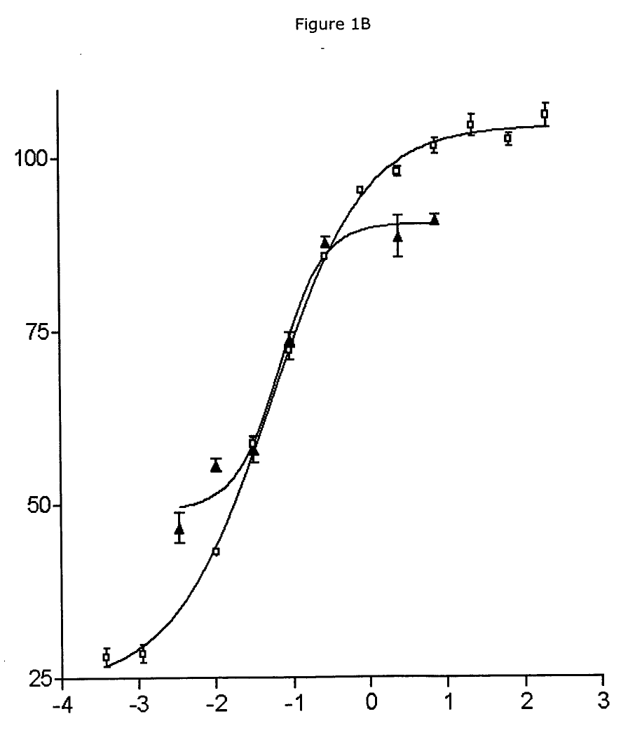

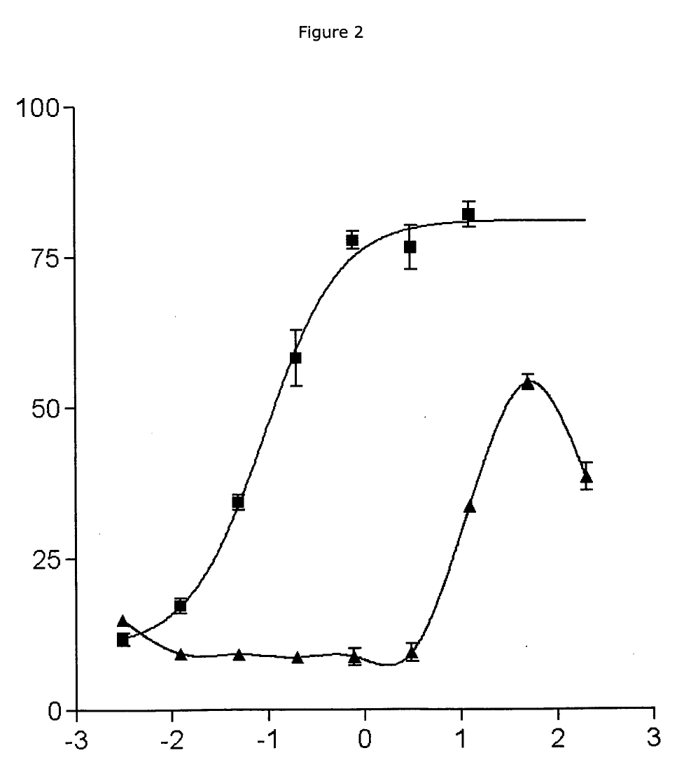

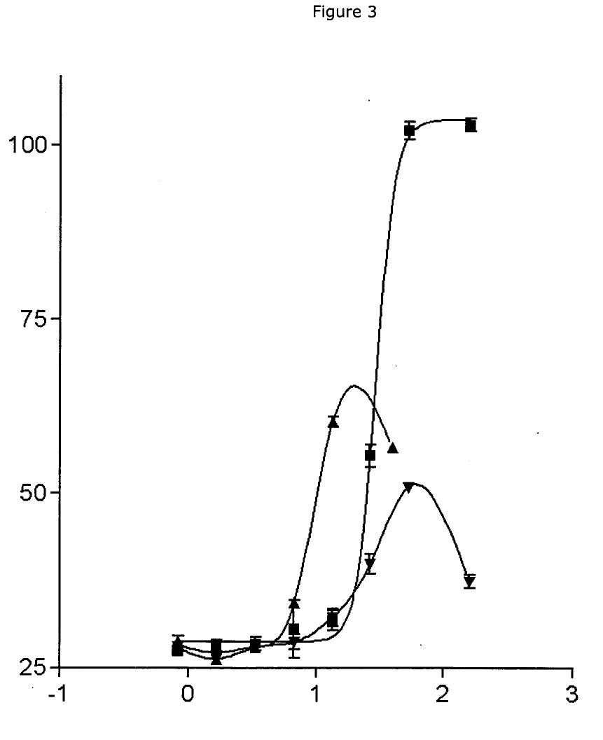

We found a specific saturable recruitment of co-factors to LXRα and LXRβ after addition of FF-MAS with EC₅₀ values of about 21 nM and about 67 nM for LXRβ and LXRα, respectively. The EC₅₀ value is the dose which leads to a 50% saturation. These data (FIGS. 1-3) show that LXR is a signalling partner or receptor for FF-MAS and is involved in maturation of oocytes.

### EXAMPLE 5

Both LXR═ and LXR mRNA (found by RT-PCR) and protein (visualized by IHC) are expressed in mouse oocytes.

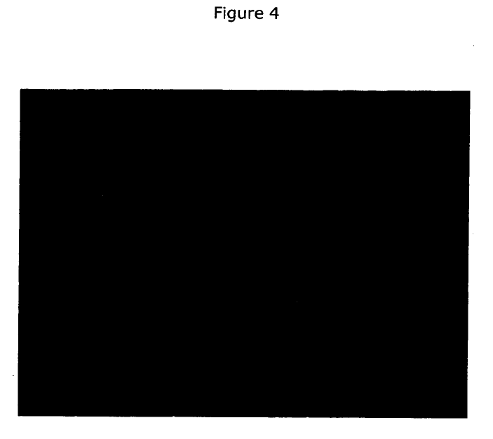

Example: LXRα in the mouse oocyte (FIG. 4).

1. A method for determining whether a compound that has an EC₅₀ value above 500 nM in a homogenous time-resolved fluorescence assay exhibits meiosis activating activity, said method comprising testing the effect of said compound on germinal vesicle breakdown (GVBD) in an oocyte maturation assay, whereby measurement of a GVBD value for said compound in said oocyte maturation assay that is greater than the GVBD value measured in said oocyte maturation assay in the absence of said compound indicates that said compound has meiosis activating activity.

2. The method of claim 1, wherein said compound having meiosis activating activity is useful as fertility promoting compound.

3. A method for determining whether a compound that has an EC₅₀ value greater than 500 nM in a homogenous time-resolved fluorescence assay inhibits meiosis activating activity, said method comprising testing the effect of said compound on germinal vesicle breakdown (GVBD) in the presence of 4,4-dimethyl-5α-cholesta-8,14,24-triene-3β-ol (FF-MAS) in an oocyte maturation assay, whereby measurement of a GVBD value for said compound in said oocyte maturation assay that is less than the GVBD value measured in said oocyte maturation assay in the absence of said compound indicates that said compound inhibits meiosis activating activity.

4. The method of claim 3, wherein said compound that inhibits meiosis activating activity is useful as a contraceptive.

5. A method for detecting the presence of an antagonist of a meiotic regulating substance (MAS), said method comprising the steps of a) exposing a compound in the presence of a MAS agonist to a nuclear hormone receptor (NR) coupled to a response pathway under conditions and for a time sufficient to allow binding of the compound to the NR and an associated response through the pathway; and b) detecting a reduction in the stimulation of the response pathway resulting from the binding of the compound to the NR, relative to the stimulation of the response pathway by the MAS agonist alone and therefrom determining the presence of a MAS antagonist.

6. The method of claim 5, wherein said NR is a Liver-X-Receptor (LXR).

7. The method of claim 6, wherein the LXR is LXRα.

8. The method of claim 6, wherein the LXR is LXRβ.

9. A method for detecting the presence of an agonist of a meiotic regulating substance (MAS), said method comprising the steps of a) exposing a compound to a nuclear hormone receptor (NR) coupled to a response pathway under conditions and for a time sufficient to allow binding of the compound to the NR and an associated response through the pathway; and b) detecting a stimulation of the response pathway resulting from the binding of the compound to the NR, relative to the stimulation of the response pathway detected in the absence of said compound and therefrom determining the presence of a MAS agonist.

10. The method of claim 9, wherein said NR is a Liver-X-Receptor (LXR).

11. The method of claim 10, wherein the LXR is LXRα.

12. The method of claim 10, wherein the LXR is LXRβ.

13. A method of screening for agonists or antagonists of 4,4-dimethyl-5α-cholesta-8,14,24-triene-3β-ol (FF-MAS) activity, the method comprising incubating a nuclear hormone receptor (NR) with a substance suspected to be an agonist or antagonist of FF-MAS, and subsequently with FF-MAS or an analogue thereof, and detecting any effect of binding of FF-MAS or the analogue to meiotic regulating substance (MAS) receptor.

14. A method of screening for agonists or antagonists of 4,4-dimethyl-5α-cholesta-8,14,24-triene-3β-ol (FF-MAS) activity, the method comprising incubating FF-MAS or an analogue thereof with a substance suspected to be an agonist or antagonist of activity of FF-MAS and subsequently with a nuclear hormone receptor (NR), and detecting any effect of binding of FF-MAS or the analogue to the NR.
